(19) United States
(12) Patent Application Publication
(10) Pub. No.: US 2023/0348973 A1
(43) Pub. Date: Nov. 2, 2023

(54) PAIRED-END RE-SYNTHESIS USING BLOCKED P5 PRIMERS

(71) Applicant: ILLUMINA CAMBRIDGE LIMITED, Cambridge (GB)

(72) Inventors: Xiaoyu Ma, San Diego, CA (US); Mathieu Lessard-Viger, San Diego, CA (US); Eric Brustad, San Diego, CA (US); Jeffrey Fisher, San Diego, CA (US); Jonathan Boutell, Bishops Stortford (GB); Weihua Chang, San Diego, CA (US)

(73) Assignee: ILLUMINA CAMBRIDGE LIMITED, Cambridge (GB)

(21) Appl. No.: 18/193,480

(22) Filed: Mar. 30, 2023

Related U.S. Application Data
(60) Provisional application No. 63/325,752, filed on Mar. 31, 2022.
(60) Provisional application No. 63/392,225, filed on Jul. 26, 2022.

(51) Int. Cl. C12Q 1/6874; C12Q 1/6853

(52) U.S. Cl. CPC C12Q 1/6874; C12Q 1/6853

(57) ABSTRACT

The present disclosure is generally directed to strategies for template capture and amplification during sequencing. In some examples, a solid support is used for template capture and amplification.

### CROSS-REFERENCE TO RELATED APPLICATIONS

This application claims the benefit of U.S. Provisional Patent Application No. 63/325,752, filed Mar. 31, 2022, and entitled “Paired-End Re-Synthesis Using Blocked P5 Primers,” and U.S. Provisional Patent Application No. 63/392,225, filed Jul. 26, 2022, and entitled “Paired-End Re-Synthesis Using Blocked P5 Primers,” the entire contents of each of which are incorporated by reference herein.

### FIELD

The present disclosure is generally directed to strategies for template capture and amplification during sequencing.

### SEQUENCE LISTING

The instant application contains a Sequence Listing which has been submitted electronically in xml format and is hereby incorporated by reference in its entirety. Said xml copy was created on Feb. 14, 2023, is named IP-2288-US.xml, and is 21.3 kilobytes in size.

### BACKGROUND

The detection of analytes such as nucleic acid sequences that are present in a biological sample has been used as a method for identifying and classifying microorganisms, diagnosing infectious diseases, detecting and characterizing genetic abnormalities, identifying genetic changes associated with cancer, studying genetic susceptibility to disease, and measuring response to various types of treatment. A common technique for detecting analytes such as nucleic acid sequences in a biological sample is nucleic acid sequencing.

Advances in the study of biological molecules have been led, in part, by improvement in technologies used to characterise the molecules or their biological reactions. In particular, the study of the nucleic acids DNA and RNA has benefited from developing technologies used for sequence analysis.

Paired end (PE) re-synthesis enables the sequencing of both ends of the DNA fragments, such that there are two reads, but Read 2 (R2) features weakened properties comparing to the Read 1 in terms of quality and intensity. PE always requires multiple cycles' template amplification prior to processing Read 2 to obtain the proper sequencing intensity. Disclosed herein are compositions and methods to accelerate PE re-synthesis, as well as improve R2 sequence quality.

### SUMMARY

Some examples herein provide a method of sequencing a target nucleic acid sequence, wherein the method includes; providing a solid support having immobilised thereon a cluster of first immobilised nucleic acid strands including a target nucleic acid sequence, wherein the solid support has a plurality of dormant lawn primers, wherein the dormant lawn primers are blocked at the 3′ end; carrying out a first sequencing read to determine the sequence of a region of the first immobilised strands, preferably by a sequencing-by-synthesis technique or by a sequencing-by ligation technique; removing the blocking groups from the dormant primers to allow extension from the 3′ ends of the unblocked primers; carrying out an extension reaction to extend the unblocked primer using the immobilised strand as a template to generate a cluster of second immobilised nucleic acid strands; carrying out a second sequencing read to determine the sequence of a region of the second immobilised strands, preferably by a sequencing-by-synthesis technique or by a sequencing-by ligation technique. Determining the sequences of the regions of the first and second immobilised strands achieves pairwise sequencing of said target nucleic acid sequence.

In some examples, the blocking group is a phosphate group that is bound to the 3′ end of the dormant lawn primers. In some examples, the method further includes at least one step of amplifying the immobilised target nucleic acids, for example using bridge amplification.

Some examples herein provide a solid support for use in sequencing, wherein the support includes a plurality of lawn primers immobilised on the solid support, and a plurality of dormant lawn primers immobilised on the solid support, wherein the dormant lawn primers includes a blocking 3′ group that prevents extension until removed.

In some examples, the plurality of lawn primers includes a P5 primer and a P7 primer. In some examples, the P5 primer includes a nucleic acid sequence as defined in any one of SEQ ID NO: 6, SEQ ID NO: 7, SEQ ID NO: 8, SEQ ID NO: 9, SEQ ID NO: 10, or SEQ ID NO: 11, or a variant thereof, and wherein the P7 primer includes a nucleic acid sequence as defined in SEQ ID NO: 2 or a variant thereof. In some examples, P5 is between 10 and 50 pb, and P7 is between 10 and 50 bp. In some examples, P5 is between 5 and 25 bp, and P7 is between 5 and 25 bp. In some examples, P5 is 9 bp, 10 bp or 13 bp, and P7 is 9 pb, 10 bp, or 13 bp.

In some examples, the plurality of dormant lawn primers includes a BsP5 primer. In some examples, the dormant lawn primers includes at least 80%, at least 85%, at least 90%, or at least 95% of any of SEQ ID NO: 3, SEQ ID NO: 4, or SEQ ID NO: 5.

In some examples, the dormant lawn primers are blocked at the 3′ end by a phosphate group.

In some examples, the lawn:dormant lawn primer ratio is selected from about 5:1, about 4:1, about 3:1, about 2:1, about 1:1, about 1:2, about 1:3, about 1:4 and about 1:5.

Some examples herein provide a solid support comprising a plurality of lawn primers including at least one P5 primer and at least one P7 primer, and a plurality of dormant lawn primers including at least one P5 primer.

In some examples, at least one dormant lawn P5 primer is blocked at the 3′ end. In some examples, the at least one of P5 primers is blocked at the 3′ end with a phosphate group.

In some examples, the at least one P7 lawn primer, the least one P5 lawn primer, and the least one P5 dormant lawn primer exist in approximately the following ratio: 1 (P7 lawn primer): 1 (P5 lawn primer): 2 (P5 dormant lawn primer).

In some examples, the at least one P5 dormant lawn primer includes at least 80%, at least 85%, at least 90%, or at least 95% sequence identity to any of SEQ ID NO: 3, SEQ ID NO: 4, or SEQ ID NO: 5.

Some examples herein provide a method of amplifying a nucleic acid template, wherein the method includes (i) applying a nucleic acid template library in solution to a solid support; wherein the template library includes a plurality of template strands, wherein each template strand includes a first or second 5′ primer-binding sequence and a first or second 3′ primer binding sequence; and wherein the solid support has immobilised thereon a plurality of lawn primer sequences complementary to the 3′ primer-binding sequence, wherein the plurality of lawn primers includes at least one P7 lawn primer, at least P5 lawn primer, and at least one dormant P5 lawn primer that include a blocking group on its 3′ end; (ii) hybridising the first or second 3′ primer binding sequence of the single stranded template strand to a lawn primer; (iii) carrying out an extension reaction to extend the lawn primer to generate a first immobilised strand complementary to the template strand, wherein the immobilised strand includes a 3′ primer binding sequence; (iv) displacing the template strand from the first immobilised strand and amplifying the first immobilised strand; (v) linearizing the amplified immobilised strand; (vi) removing the blocking group from the 3′ end of the at least one dormant P5 primer; and (vii) amplifying the linearized immobilised strand. In some examples, the amplifying steps may be performed using bridge amplification.

In some examples, the blocking group on the 3′ end of the least one dormant P5 lawn primer includes a phosphate group.

In some examples, the P5 lawn primer includes a nucleic acid sequence as defined in any one of SEQ ID NO: 6, SEQ ID NO: 7, SEQ ID NO: 8, SEQ ID NO: 9, SEQ ID NO: 10, or SEQ ID NO: 11, or a variant thereof, and the P7 lawn primer includes a nucleic acid sequence as defined in SEQ ID NO: 2 or a variant thereof.

In some examples, the P5 lawn primer is between 5 and 25 bp, and the P7 lawn primer is between 5 and 25 bp. In some examples, the P5 lawn primer is 10 bp, 13 bp or 15 bp, and the P7 lawn primer is 10 bp, 13 bp, or 15 bp.

In some examples, removing the blocking group from the 3′ end of the at least one dormant P5 primer includes using a phosphatase. In some examples, the ratio of lawn:dormant lawn primers is selected from about 5:1, about 4:1,about 3:1, about 2:1, about 1:1, about 1:2, about 1:3, about 1:4, and about 1:5.

Some examples herein provide a method of sequencing immobilised nucleic acids, wherein the method includes a solid support having a cluster of first immobilised nucleic acids bound to P5 lawn primers, and blocked P5 lawn primers; cleaving the P5 lawn primers to allow linearization of the first immobilised nucleic acids; carrying out a first extension reaction to determine a sequence of a region of the first immobilised nucleic acids; removing the blocking groups from the blocked P5 lawn primers resulting in unblocked P5 lawn primers; carrying out a second extension reaction to extend the unblocked P5 lawn primers using the first immobilised nucleic acids as a template to generate a cluster of second immobilised nucleic acids; carrying out a second extension reaction to determine the sequence of a region of the second immobilised nucleic acids; wherein determining the sequences of the regions of the first and second immobilised nucleic acids achieves pairwise sequencing of said target nucleic acid sequence.

In some examples, cleaving the P5 lawn primers takes place at the 5′ end of the P5 lawn primers. In some examples, cleaving the P5 lawn primers takes place upstream of a region of the P5 lawn primers that bind to the first immobilised nucleic acids. In some examples, cleaving the P5 lawn primers takes place in a spacer region of the P5 lawn primers. In some examples, the spacer region of the P5 lawn primers is upstream of a region of the P5 lawn primers that bind to the first immobilised nucleic acid strands. In some examples, the blocked P5 primers including blocking groups at the 3′ end of the blocked P5 primers. In some examples, removing the blocking groups includes removing the phosphate groups.

Some examples herein provide a composition, having a solid support having a cluster of immobilised nucleic acids bound to unblocked P5 lawn primers, wherein the unblocked P5 lawn primers including a spacer region on their 5′ end; and blocked P5 lawn primers, wherein the blocked P5 lawn primers include blocking groups on their 3′ end.

In some examples, the spacer region includes one or more thymine nucleobases. In some examples, the spacer region includes six (6) thymine nucleobases. In some examples, the unblocked P5 lawn primers are capable of being cleaved at any one or more of the six (6) thymine nucleobases. In some examples, the unblocked P5 lawn primers have a sequence having at least 80%, at least 85%, at least 90%, or at least 95% sequence identity with any one of SEQ ID NOs: 7-11. In some examples, the unblocked P5 lawn primers include any one of SEQ ID NOs: 7-11. In some examples, the blocking groups contain phosphate groups.

Some examples herein provide a method of sequencing a target nucleic acid including generating an immobilised cluster of first nucleic acid strands on a solid support, wherein a plurality of p7 lawn primers and a plurality of blocked P5 lawn primers are immobilised on the solid support, and wherein the plurality of blocked P5 lawn primers have been doped with a solution comprising a single-stranded binding protein; carrying out a first sequencing read to determine a sequence of a region of the first immobilised nucleic acid strands; removing the blocking groups from the P5 primers to allow extension from the 3′ ends of the unblocked P5 primers; carrying out a second extension reaction to extend the unblocked P5 primers using the immobilised nucleic strands as a template to generate a cluster of second immobilised nucleic acid strands; carrying out a second sequencing read to determine a sequence of a region of the second immobilised strands; wherein determining the sequences of the regions of the first and second immobilised nucleic acid strands achieves pairwise sequencing of said target nucleic acid sequence.

In some examples, the single-stranded binding protein includes gp32.

In some examples, the concentration of the single-stranded binding protein in the solution is greater than or equal to 39 uM and less than or equal to 45 uM. In some examples, a concentration of the single-stranded binding protein that is greater than or equal to 39 uM and less than or equal to 45 uM functions to inhibit or prevent consumption of the single-stranded binding protein by the blocked P5 primers.

In some examples, the solution further includes a recombinase.

In some examples, the concentration of the recombinase is greater than or equal to 5.6 uM and less than or equal to 7.6 uM. In some examples, a concentration of the recombinase that is greater than or equal to 5.6 uM and less than or equal to 7.6 uM functions to inhibit or prevent consumption of the recombinase by the blocked P5 primers.

Some examples herein provide a solid support, where the solid support includes a plurality of P7 primers immobilised on the solid support; and a plurality of blocked P5 primers immobilised on the solid support, where the blocked P5 primers have been doped with a solution comprising a single-stranded binding protein.

In some examples, the solution further includes a recombinase.

In some examples, the concentration of the single-stranded binding protein in the solution is greater than or equal to 39 uM and less than or equal to 45 uM.

In some examples, the concentration of recombinase is greater than or equal to 5.6 uM and less than or equal to 7.6 uM,

Some examples herein provide a method, including doping a plurality of P5 lawn primers with a solution comprising a single-stranded binding protein and a recombinase, where the concentration of the single-stranded binding protein is greater than or equal to 41 uM and less than or equal to 43 uM, and where the concentration of the recombinase is greater than or equal to 5.6 uM and less than or equal to 7.6 uM.

In some examples, the single-stranded binding protein includes gp32.

In some examples, the method further includes immobilising the doped P5 lawn primers on a solid support.

In some examples, the method further includes immobilising P7 lawn primers on a solid support.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF THE DRAWINGS

FIG. 1 shows a schematic paired end (PE) re-synthesis method which utilizes blocked, short P5 primers (BsP5) that are added to the solid support prior to DNA clustering.

FIG. 2 includes various full P5 sequences with a CCL cleavage position within T spacers (respectively SEQ ID NOS: 6, 7, 8, 9, 10, and 11).

FIGS. 3A-3C show the signal intensity for fast paired end turn using ExAmp with one push for 5 min. (FIG. 3A) shows the standard conditions for ExAmp as a control, (FIG. 3B) shows non-bridging clustering using the 10 base pair, blocked, short P5 primer (BsP5) (FIG. 3C) shows non-bridging clustering using the 13 base pair, blocked, short P5 primer (BsP5).

FIG. 4 shows the ratio between lawn-P7 primer binding sequences and the dormant-P5s affects the R1 and R2 intensity. A higher concentration of BsP5 results in better PE turn but lower R1 intensity (P7: 1.1 uM; ExAmp: Ras6T; Library: N450 at 200 μM).

FIG. 5 shows that the decrease in R1 intensity when using BsP5 is probably due to unwanted annealing with the templates. However, further shortening of the length of BsP5 can be used to further lower the Tm and inhibit unwanted annealing.

FIG. 6 is a schematic of generation of a single-stranded library from a double-stranded template library.

FIGS. 7A-7B show data comparing sequencing metrics between a standard clustering workflow and a non-bridging clustering (NBC) workflow, using two different formulations (Ras6T (see Table 1) (FIG. 7A)) and TCX (see Table 2 (FIG. 7B)) for doping the P5 primers.

### DETAILED DESCRIPTION

The following features apply to all aspects of the present disclosure.

Sequencing generally includes four fundamental steps: 1) library preparation to form a plurality of template molecules available for sequencing; 2) cluster generation to form an array of amplified single template molecules on a solid support; 3) sequencing the cluster array; and 4) data analysis to determine the target sequence.

Library preparation is the first step in any high-throughput sequencing platform. During library preparation, nucleic acid sequences, for example genomic DNA sample, or cDNA or RNA sample, is converted into a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adapters (adapter sequences). This may be followed by amplification and sequencing. The original sample DNA fragments are referred to as “inserts.” Alternatively “tagmentation” can be used to attach the sample DNA to the adapters. In tagmentation, double-stranded DNA is simultaneously fragmented and tagged with adapter sequences and PCR primer binding sites. The combined reaction eliminates the need for a separate mechanical shearing step during library preparation. The target polynucleotides may advantageously also be size-fractionated prior to modification with the adaptor sequences.

### Terms

As used herein an “adapter” sequence includes a short sequence-specific oligonucleotide that is ligated to the 5′ and 3′ ends of each DNA (or RNA) fragment in a sequencing library as part of library preparation. The adaptor sequence may further include non-peptide linkers.

The term “cluster” refers to a discrete site on a solid support that include a plurality of identical immobilised nucleic acid strands.

By “complementary” is meant that the primer has a sequence of nucleotides that can form a double-stranded structure by matching base-pairs with the adaptor or primer sequence or part thereof. By “substantially complementary” is meant that the primer has at least 85%, 90%, 95%, 98%, 99% or 100% overall sequence identical to the complementary sequence.

The terms “hybridise” and “anneal” can be used interchangeably. In one example, hybridisation occurs under SXSSC (saline sodium citrate) at 38° C.

The term “variant” as used herein with reference to any of the sequences recited herein refers to a variant nucleic acid that is substantially identical, i.e. has only some sequence variations, for example to the non-variant sequence. In one example, a variant has at least 50%, 55%, 60%, 65%, 70%, 75%, 80%, 85%, 86%, 87%, 88%, 89%, 90%, 91%, 92%, 93%, 94%, 95%, 96%, 97%, 98%, or at least 99% overall sequence identity to the non-variant nucleic acid sequence.

As used herein, the term “solid support” refers to a rigid substrate that is insoluble in aqueous liquid. The substrate can be non-porous or porous. The substrate can optionally be capable of taking up a liquid (e.g. due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fibre bundles, and polymers. A particularly useful material is glass. Other suitable substrate materials may include polymeric materials, plastics, silicon, quartz (fused silica), boro float glass, silica, silica-based materials, carbon, metals including gold, an optical fibre or optical fibre bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength, such as one or more of the techniques set forth herein. Conversely, it may be desirable to select a material that does not pass radiation of a certain wavelength (e.g. being opaque, absorptive or reflective). This can be useful for formation of a mask to be used during manufacture of the structured substrate; or to be used for a chemical reaction or analytical detection carried out using the structured substrate. Other properties of a material that can be exploited are inertness or reactivity to certain reagents used in a downstream process; or ease of manipulation or low cost during a manufacturing process manufacture. Further examples of materials that can be used in the structured substrates or methods of the present disclosure are described in U.S. Ser. No. 13/661,524 and US Pat. App. Pub. No. 2012/0316086 A1, the entire contents of each are incorporated by reference herein.

The term “re-synthesis” means a primer that is capable of synthesising the reverse or complement strand after the first sequencing read (i.e. read 1).

As used herein, the term “re-synthesis primer” is also referred to herein as a dormant lawn primer, and such terms may be used interchangeably.

As used herein, the term “TCX” refers to a formulation for doping BsP5 primers. Its components are described in Table 1.

As used herein, the term “Ras6T” refers to a formulation for doping BsP5 primers. Its components are described in Table 2.

As used herein, the term “percentage pass filter” or “% pass filter” refers to the signal purity that is generated from a clonal grouping of template nucleic acids that is generated after a sequencing run. A “percentage pass filter” of 70 or more indicates high signal purity and, thus, a successful sequencing run.

As used herein, the term “doping” refers to a process in which primers are blocked such that they cannot participate in cluster generation. In some examples, “doping” primers results in blocking the primers at their 3′ end. As used herein, the term “doped” refers to primers that have been blocked through the process of “doping.”

### Description of Examples of the Disclosure

In some examples, paired end (PE) re-synthesis is achieved using “blocked” or “dormant” lawn primers. These primers do not participate in cluster generation but only in re-synthesis prior to sequencing. In some examples, the lawn primer is blocked at the 3′ end, which is removed prior to re-synthesis—e.g. following generation of the cluster. In this way the lawn primer can be considered dormant until the sequencing step. The 3′ block may be a phosphate group or another reversible blocking group. In some examples, the PE re-synthesis method includes at least one step of amplification of the targets, for example using bridge amplification.

Some examples herein provide a method that includes the follow steps: (i) amplification of templates on a solid support that includes lawn primers that bind to the templates to carry out the amplification, and dormant lawn primers that are blocked at their 3′ end; (ii) linearization of the templates; (iii) de-protection of the 3′ end of the dormant lawn primers; and (iv) re-synthesis in which the de-protected dormant lawn primers can now hybridize to the templates and be extended based on the sequence of the templates.

In some examples, the amplification may be performed using bridge amplification, while in other examples the amplification may be performed using recombinase polymerase amplification (ExAmp).

In some examples step (i) results in cluster amplification of the templates. In some examples, because the dormant law primers are blocked at their 3′ end, they do not participate in cluster amplification of the templates. In some examples, the dormant lawn primers are blocked on their 3′ end with a phosphate group. In some examples, the dormant lawn primers include a blocked short P5 primer (BsP5). In some examples, the BsP5 does not include a cleavage site.

In some examples, linearization of the templates includes cleaving the lawn primers. In some examples, cleavage of the lawn primers takes place in the spacer region of the lawn primers. In some examples, the spacer region is on the 5′ end of the lawn primers. In some examples, the spacer region includes a Poly-T spacer.

In some examples, cleaving the lawn primers results in fragment nucleotide sequences that are too small to bind to the templates. In some examples, cleaving the lawn primers results in a fragment nucleotide sequence that is less than twenty (20) nucleotides, or less than ten (10) nucleotides, or less than seven (7) nucleotides. In some examples, the fragment nucleotide sequence is a string of six (6) or less thymine nucleotides.

In some examples, the size of the cleaved lawn primers combined with the de-protected dormant lawn primers, results in selective hybridization of the templates to the de-protected dormant lawn primers.

Some examples herein provide a method of sequencing a target nucleic acid sequence, wherein the method includes, providing a solid support having immobilised thereon a cluster of first immobilised nucleic acid strands including a target nucleic acid sequence, wherein the solid support has a plurality of dormant lawn primers, wherein the dormant lawn primers are blocked at the 3′ end; carrying out a first sequencing read to determine the sequence of a region of the first immobilised strands, preferably by a sequencing-by-synthesis technique or by a sequencing by-ligation technique; removing the blocking groups from the dormant primers to allow extension from the 3′ ends of the unblocked primers; carrying out an extension reaction to extend the unblocked primer using the immobilised strand as a template to generate a cluster of second immobilised nucleic acid strands; carrying out a second sequencing read to determine the sequence of a region of the second immobilised strand, preferably by a sequencing-by-synthesis technique or by a sequencing-by ligation technique, wherein determining the sequences of the regions of the first and second immobilised strands achieves pairwise sequencing of said target nucleic acid sequence.

In some examples, the blocking group includes a phosphate group that is bound to the 3′ end of the dormant lawn primers. In some examples, the blocking group includes any blocking agent on the 3′ end of the dormant lawn primers that is capable of preventing binding to the dormant lawn primers. In some examples, the blocking agent includes a hairpin structure.

In some examples, the dormant lawn primers include P5 primers. In some examples, the dormant lawn primers include short P5 primers. In some examples, the short P5 primers are less than 10 bp.

In some examples, the method further includes at least one step of amplifying the immobilised target nucleic acids. The process of amplification can occur through any of the bridge amplification methods described herein, or other suitable amplification method such as ExAmp. In some examples, at least one of the amplification steps occurs after removal of the blocking group from the dormant primers.

Some examples herein provide a solid support for use in sequencing, wherein the support includes a plurality of lawn primers immobilised on the solid support, and a plurality of dormant lawn primers immobilised on the solid support, wherein the dormant lawn primers include a blocking 3′ group that prevents extension until removed. In some examples, the blocking 3′ group of the dormant primers is removed to allow for amplification of the template strands, for example using bridge amplification.

In some examples, the plurality of lawn primers includes any one or more of P5 and a P7 primer, e.g., includes both P5 primers and P7 primers. In some examples, the P5 primer includes a nucleic acid sequence that includes at least 80%, at least 85%, at least 90%, or at least 95% of any one of SEQ ID NO: 6, SEQ ID NO: 7, SEQ ID NO: 8, SEQ ID NO: 9, SEQ ID NO: 10, or SEQ ID NO: 11, and the P7 primer includes a nucleic acid sequence that includes at least 80%, at least 85%, at least 90%, at least 95% of SEQ ID NO: 2. In some examples, the P5 primer includes a nucleic acid sequence as defined in any one of SEQ ID NO: 6, SEQ ID NO: 7, SEQ ID NO: 8, SEQ ID NO: 9, SEQ ID NO: 10, or SEQ ID NO: 11, or a variant thereof, and the P7 primer includes a nucleic acid sequence as defined in SEQ ID NO: 2, or a variant thereof.

In some examples, the P5 primer (e.g., including any one of SEQ ID NO: 6, SEQ ID NO: 7, SEQ ID NO: 8, SEQ ID NO: 9, SEQ ID NO: 10, or SEQ ID NO: 11 or a variant thereof) is between 10 and 50 pb, and the P7 primer (e.g., including SEQ ID NO: 2 or a variant thereof) is between 10 and 50 bp. In some examples, the P5 primer is between 5 and 25 bp, and the P7 primer is between 5 and 25 bp. In some examples, the P5 primer is 9 bp, 10 bp or 13 bp, and the P7 primer is 9 pb, 10 bp, or 13 bp. In some examples, the P5 primer is greater than 50 bp, and the P7 primer is greater than 50 bp.

In some examples, the plurality of dormant lawn primers includes BsP5 primer. In some examples, the dormant lawn primers include a sequence that is at least 80%, at least 85%, at least 90%, or at least 95% of any of SEQ ID NO: 3, SEQ ID NO: 4, or SEQ ID NO: 5. In some examples, the dormant lawn primers include any of the sequences of SEQ ID NO: 3, SEQ ID NO: 4, or SEQ ID NO: 5.

In some examples, the dormant lawn primers are blocked at the 3′ end by a phosphate group. In some examples, the dormant lawn primers are blocked by any agent capable of blocking the 3′ end of the dormant lawn primers. In some examples, the agent includes a hairpin structure.

In some examples, the lawn:dormant lawn primer ratio is selected from about 5:1, about 4:1, about 3:1, about 2:1, about 1:1, about 1:2, about 1:3, about 1:4 and about 1:5.

Some examples herein provide a solid support comprising a plurality of lawn primers including at least one P5 primer and at least one P7 primer, and a plurality of dormant lawn primers including at least one P5 primer.

In some examples, at least one dormant lawn P5 primer is blocked at the 3′ end. In some examples, the at least one P5 primer is blocked at the 3′ end with a phosphate group. In some examples, the at least one P5 primer is blocked with any agent capable of blocking the 3′ end of the P5 primer. In some examples, the agent includes a hairpin structure.

In some examples, the at least one P7 lawn primer, the least one P5 lawn primer, and the least one P5 dormant lawn primer exist in approximately the following ratio: 1 (P7 lawn primer): 1 (P5 lawn primer): 2 (P5 dormant lawn primer).

In some examples, the at least one P5 dormant lawn primer includes at least 80%, at least 85%, at least 90%, or at least 95% sequence identity to any of SEQ ID NO 3; SEQ ID NO: 4, or SEQ ID NO: 5.

Some examples herein provide a method of amplifying a nucleic acid template, wherein the method includes (i) applying a nucleic acid template library in solution to a solid support; wherein the template library includes a plurality of template strands, wherein each template strand includes a first or second 5′ primer-binding sequence and a first or second 3′ primer binding sequence; and wherein the solid support has immobilised thereon a plurality of lawn primer sequences complementary to the 3′ primer-binding sequence, wherein the plurality of lawn primers includes at least one P7 lawn primer, at least P5 lawn primer, and at least one dormant P5 lawn primer that includes a blocking group on its 3′ end; (ii) hybridising the first or second 3′ primer binding sequence of the single stranded template strand to a lawn primer; (iii) carrying out an extension reaction to extend the lawn primer to generate a first immobilised strand complementary to the template strand, wherein the immobilised strand includes a 3′ primer binding sequence; (iv) displacing the template strand from the first immobilised strand and amplifying the extended immobilised strand; (v) linearizing the amplified immobilised strand; (vi) removing the blocking group from the 3′ end of the at least one dormant P5 primer; (vii) amplifying the linearized immobilised strand. In some examples, the amplification is performed using bridge amplification.

In some examples, the blocking group on the 3′ end of the least one dormant P5 lawn primer includes a phosphate group. In some examples, the blocking group includes any agent capable of blocking the 3′ end of the dormant lawn primer. In some examples, the agent includes a hairpin structure.

In some examples, the steps of amplification can include any of the bridge amplification methods described herein, or other suitable amplification method such as ExAmp.

In some examples, the P5 lawn primer includes a nucleic acid sequence as defined in any one of SEQ ID NO: 6, SEQ ID NO: 7, SEQ ID NO: 8, SEQ ID NO: 9, SEQ ID NO: 10, or SEQ ID NO: 11, or a variant thereof, and P7 lawn primer includes a nucleic acid sequence as defined in SEQ ID NO: 2 or a variant thereof.

In some examples, the P5 lawn primer is between 5 and 25 bp, and the P7 lawn primer is between 5 and 25 bp. In some examples, the P5 lawn primer is 10 bp, 13 bp or 15 bp, and the P7 lawn primer is 10 bp, 13 bp, or 15 bp.

In some examples, removing the blocking group from the 3′ end of the at least one dormant P5 primer includes using a phosphatase. In some examples, the ratio of lawn:dormant lawn primers is selected from about 5:1, about 4:1, about 3:1, about 2:1, about 1:1, about 1:2, about 1:3, about 1:4, and about 1:5.

Some examples herein provide a method of sequencing immobilised nucleic acids, wherein the method includes using a solid support having a cluster of first immobilised nucleic acids bound to P5 lawn primers, and blocked P5 lawn primers; cleaving the P5 lawn primers to allow linearization of the first immobilised nucleic acids; carrying out a first extension reaction to determine a sequence of a region of the first immobilised nucleic acids; removing the blocking groups from the blocked P5 lawn primers resulting in unblocked P5 lawn primers; carrying out a second extension reaction to extend the unblocked P5 lawn primers using the first immobilised nucleic acids as a template to generate a cluster of second immobilised nucleic acids; carrying out a second extension reaction to determine the sequence of a region of the second immobilised nucleic acids; wherein determining the sequences of the regions of the first and second immobilised nucleic acids achieves pairwise sequencing of said target nucleic acid sequence.

In some examples, cleaving the P5 lawn primers takes place at the 5′ end of the P5 lawn primers. In some examples, cleaving the P5 lawn primers takes place upstream of a region of the P5 lawn primers that bind to the first immobilised nucleic acids. In some examples, cleaving the P5 lawn primers takes place in a spacer region of the P5 lawn primers. In some examples, the spacer region of the P5 lawn primers is upstream of a region of the P5 lawn primers that bind to the first immobilised nucleic acid strands.

In some examples, the P5 lawn primers are cleaved on any one of the first twenty (20) bases, or the first ten (10), or first six (6) bases of the 5′ end of the primers. In some examples, the first twenty (20) bases, or the first ten (10) bases, or the first six (6) bases of the 5′ end of the P5 lawn primers includes at least three (3), at least four (4), at least five (5), or at least (6) thymine bases. In some examples, the P5 lawn primers are cleaved at a thymine base.

In some examples, the method of sequencing immobilised nucleic acids further includes creating a double stranded nucleotide sequence that results from the first extension reaction. In some examples, the method further includes removing the original template strand of the immobilised nucleic acid of the double stranded nucleotide sequence.

In some examples, the blocked P5 primers include blocking groups at the 3′ end of the blocked P5 primers. In some examples, removing the blocking groups includes removing the phosphate groups.

In some examples, the solid support used in the method of sequencing immobilised nucleic acids includes a flow cell. In some examples, the immobilised nucleic acids include one or more adaptor sequences that are complementary to one or more of the P5 lawn primers or blocked P5 lawn primers.

In some examples, any one or more of the extension reactions in the method of sequencing immobilised nucleic acids includes bridge amplification. In some examples, any one or more the extension reactions in the method of sequencing immobilised nucleic acids includes exclusion amplification (ExAmp).

Some examples herein provide a composition, having a solid support having a cluster of immobilised nucleic acids bound to unblocked P5 lawn primers, wherein the unblocked P5 lawn primers include a spacer region on their 5′ end; and blocked P5 lawn primers, wherein the blocked P5 lawn primers include blocking groups on their 3′ end.

In some examples, the spacer region includes one or more thymine nucleobases. In some examples, the spacer region includes six (6) thymine nucleobases. In some examples, the unblocked P5 lawn primers are capable of being cleaved at any one or more of the six (6) thymine nucleobases. In some examples, the unblocked P5 lawn primers have a sequence having at least 80%, at least 85%, at least 90%, or at least 95% sequence identity with any one of SEQ ID NOs: 7-11. In some examples, the unblocked P5 lawn primers include any one of SEQ ID NOs: 7-11.

In some examples, the blocked P5 lawn primers include phosphate blocked P5 primers (BsP5). In some examples, the phosphate group is on the 3′ end of the P5 primer. In some examples, the blocked P5 lawn primers is blocked through any chemical structure or any set of chemical structures that sterically hinders access to the 3′ end of the primers.

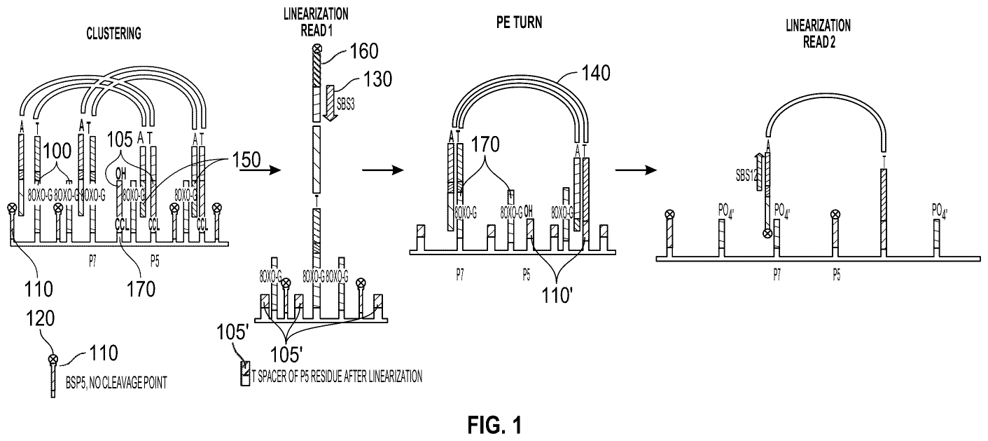

Some examples herein provide a method of sequencing as depicted in the schematic in FIG. 1. As shown in FIG. 1, there is an initial clustering step, which results in amplification of the template strands (see portion of the schematic labelled “Clustering”). Bridge amplification is illustrated in FIG. 1; however, other types of amplification can be utilized such as ExAmp.

During this initial clustering step, there are unblocked P5 lawn primers 105 and unblocked P7 lawn primers 100, and blocked or dormant lawn primers 110. The templates include adaptors 150 on their 3′ and 5′ ends that are complementary to portions of the lawn primers. In the initial clustering step, the template preferentially hybridizes to the unblocked lawn primers through complementary binding of the adaptors to the lawn primers.

The blockage of the dormant lawn primers 110 can be on the 3′ end of the primers 120. Blockage of the dormant lawn primers can occur through a phosphate being bound to the 3′ end or having the 3′ end otherwise sterically hindered.

Because the dormant lawn primers are relatively short, these primers substantially may not participate in the initial cluster formation of the templates. In particular, due to the relatively short size of the dormant lawn primers, they may lack sufficient complementarity with the adaptors of the template to substantially bind to the template. Also, the blocking group may inhibit or prevent the dormant lawn primers form participating in the clustering step, even if the dormant lawn primers hybridize to the templates. As a result, substantially only the unblocked lawn primers may participate in the initial clustering step.

After hybridization of the template to the lawn primers, the templates are amplified. The templates are extended using polymerase resulting in a double stranded nucleic acid. The hybridised template is removed leaving its complement immobilised to the surface of the flow cell via the lawn primers. The adaptor on the end of the immobilised template not bound to the lawn primers binds to another lawn primer to which it is complementary.

After the initial clustering operations, linearization of the template strand is performed, followed by sequencing, resulting in Read 1 (see portion of the schematic labelled “Linearization Read 1”). More specifically, linearization can be performed through cleavage of the unblocked P5 lawn primers 105 to form cleaved P5 lawn primers 105′. Cleavage of the unblocked lawn primers typically occurs close to the 5′ end of the primers in the T-spacer region. Once the template strand is linearized, a sequencing primer 130 can bind to the template and sequencing-by-synthesis used to sequence the template strand in a first direction.

The blocked or dormant lawn primers are then de-protected. This can be performed through removal of the phosphate group 120 or any other structure that is sterically hindering the 3′ end of the dormant lawn primers, resulting in de-protected lawn primers 110′.

This is followed by PE resynthesis in which the template is amplified. In PE resynthesis, the adaptors at the free ends of the immobilised templates 160 tend not hybridize to the cleaved lawn primers because the nucleotide length of the cleaved lawn primers are too small, and they lack sufficient sequence complementarity with the adaptors on the templates. Instead, the adaptors at the free ends of the immobilised templates hybridize to the de-protected lawn primers 110′ (see portion of schematic labelled “PE Turn”).

After hybridization there is extension and linearization of Read 2. (see portions of schematic labelled “PE Turn” and “Linearization Read 2”). The templates are extended using polymerase resulting in a double stranded nucleic acid. The hybridised template is removed leaving its complement immobilised to the surface of the flow cell via the lawn primers. The complement strand is linearized as a result of 8-oxoguanine causing lesions in complement strand 170.

The amplification steps shown in FIG. 1 can be performed through any of the bridge amplification methods described herein, or other suitable amplification method such as ExAmp.

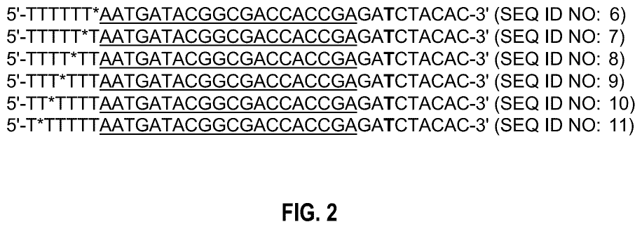

FIG. 2 shows nonlimiting examples lawn primers (e.g. P5 lawn primers 105 and P7 lawn primers 100 (see FIG. 1) including a spacer region 170 (see FIG. 1) upstream of the primer. In this case, the spacer region is a string of thymine nucleotides. The “T*” represents the site of linearization within each of the sequences. An example of a spacer region is shown as element 170 in FIG. 1. Cleavage at the linearization site would result in a short nucleotide sequence (i.e., six (6) nucleotides or less) bound to the solid support (e.g., flow cell). As a result, during PE resynthesis the adaptors at the free ends of the immobilised template strands would tend not to bind to the cleaved lawn primers and instead may hybridize to the deprotected lawn primers 110′ in a manner such as described in FIG. 1.

Linearization within the spacer region on the 5′ end of the lawn primers can be contrasted to linearization nearer to the 3′ end of the lawn primers. In FIG. 2, in each of sequences, a bolded T shows a potential linearization site, which is not preferred. Linearization at the bolded T of any of the sequences would result in lawn primers that are of a sufficient length to participate in PE resynthesis.

In some examples, the P5 dormant lawn primers are phosphate blocked P5 110 (see FIG. 1) (B-P5, also referred to herein as BsPS). In some examples, B-P5 is co-grafted with full length P7 (100) and P5 (105) prior to the DNA clustering, where the full length P7 and P5 participate in template amplification. B-P5 110 may be deprotected prior to PE re-synthesis to enable fast paired end (PE) turn and high quality Read 2. In some examples there is no linearization site on B-P5 (e.g., no CCL as may be included in full-length P5). As a result, there is no risk of incomplete linearization or linearization induced damage to B-P5. In some examples, B-P5 is designed to be relatively short, for example approximately 10 bps, resulting in less/no interference to DNA template clustering. In some examples, use of B-sP5 results in no linearization damage and fast and highly efficient PE turn re-synthesis. Also, by moving the linearization site (e.g., CCL) of full-length P5 closer to 5′ end after the poly-T spacer (e.g., as in any of SEQ ID NOS: 6-11, as compared to normal P5 shown in SEQ ID NO: 1), the DNA strands can relatively easily hybridize to B-sP5 for the re-synthesis. As used herein, “CCL” means “cluster chemical linearization” and in some examples refers to an allyl T (denoted T*) which may be cleaved using a suitable reagent formulation, e.g., a buffer solution including ethanolamine, tris(hydroxypropylphosphine) (THP), [PdAllylCl]₂, and sodium ascorbate.

The present disclosure provides a method of sequencing (e.g. by paired-end re-synthesis) that avoids damage to surface (i.e. lawn) primers (e.g. P5 lawn primers 105 and P7 lawn primers 100) during template amplification (i.e. cluster generation). This leads to more efficient PE re-synthesis. This is demonstrated by an increase in intensity of the second sequencing read (i.e. read 2).

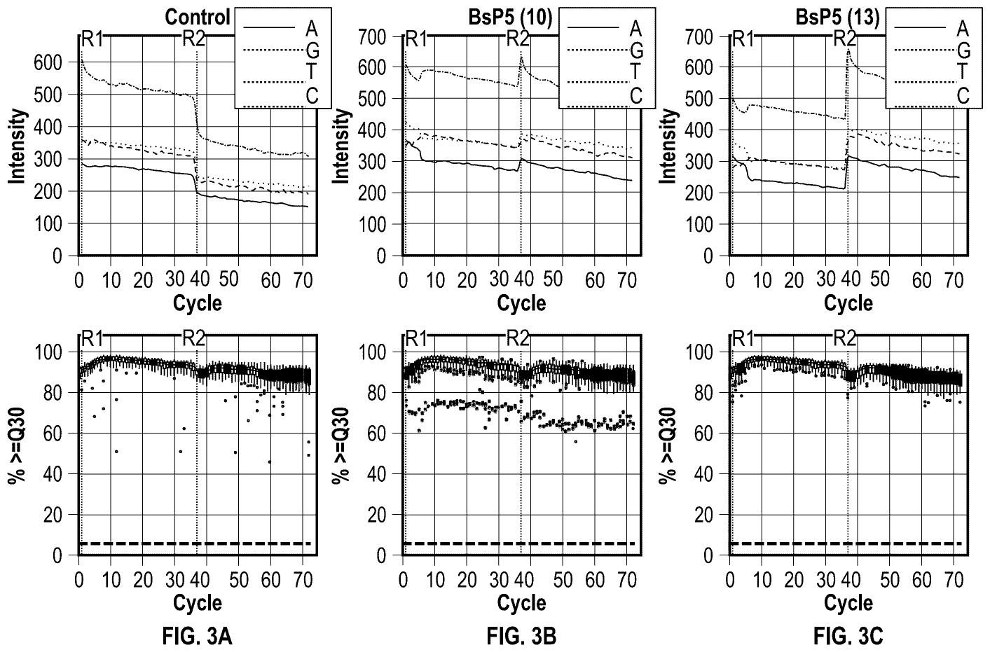

The increased efficiency of the present disclosure is further shown in FIGS. 3A-3C, which demonstrates that the use of non-bridging clustering leads to an improved signal intensity for read 2. In particular, compared to the control (FIG. 3A), non-bridging clustering in which the BsP5 primer is ten (10) base pairs (FIG. 3B) or in which the BsP5 primer is thirteen (13) base pairs (FIG. 3C), both result in higher levels of signal intensity for read 2.

In some examples, the dormant lawn primer is grafted at a concentration in the range of about 0.2 μM to about 5 μM or about 0.4 μM to about 3 μM or about 0.5 μM to about 2.5 μM. In a further example, the dormant lawn primer is grafted at about 0.5, μM or about 1.1 μM or about 2.2 μM. In a preferred example, the dormant lawn primer is grafted at about 2.2 μM.

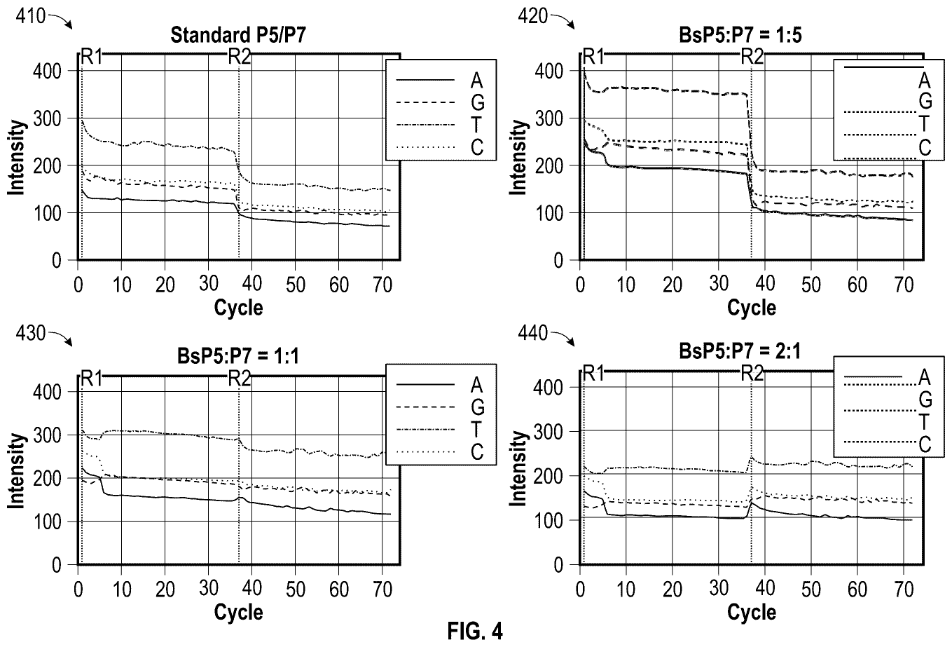

It has also been found that the ratio of lawn primers and dormant lawn primers affects read 1 and 2 intensity. As shown in FIG. 4, a higher lawn: dormant lawn primer ratio (e.g. P7 100: BsP5 110 (see FIG. 1)) leads to a high R1 intensity but lower R2 intensity (see, for example, upper right panel of FIG. 4 (420)), while a lower lawn: dormant lawn primer ratio leads to a lower R1 intensity (compared to a higher lawn: dormant lawn primer ratio) but a higher R2 intensity (see, for example, lower right panel of FIG. 4 (440))

Accordingly, in one example, the ratio of lawn:dormant lawn primer ratio is selected from about 5:1, about 4:1, about 3:1, about 2:1, about 1:1, about 1:2, about 1:3, about 1:4, and about 1:5. In a preferred example, the ratio of lawn:dormant lawn primer ratio is selected from about 2:1, about 1:1, and about 1:2. As shown in FIG. 4 a ratio of lawn:dormant lawn primers of 1:2 and 1:1 results in roughly equivalent Read 1 and Read 2 intensities (see, for example, lower panels of FIG. 4 (430 and 440)). In addition, relative to the standard P5/P7 ratio, a ratio of lawn:dormant lawn primers of 1:1 retains roughly the equivalent of the read 1 intensity but results in a significant increase of the read 2 intensity (compare, for example, the upper left panel of FIG. 4 (410) to the lower left panel of FIG. 4 (430)).

In addition, in one example, the ratio of P7 lawn primers: P5 lawn primers: BsP5 dormant primers is selected from about 0.1:0.1:2, about 0.2:0.2:2, about 0.3:0.3:2, about 0.4:0.4:2, about 0.5:0.5, about 0.6:0.6:2, about 0.7:0.7:2, about 0.8:0.8:2, about 0.9:0.9:2, about 1:1:2, about 1.1:1.1:2, about 1.2:1.2:2, about 1.3:1.3:2, about 1.4:1.4:2, about 1:5:1.5:2, about 1.6:1.6:2, about 1.7:1.7:2, about 1.8:1.8:2, about 1.9:1.9:2, about 2:2:2. Ina preferred example, the ratio of P7 lawn primers: P5 lawn primers: BsP5 dormant primers is 1:1:2.

The dormant lawn primer may be shorter in length than the full length P7 100 and P5 105 primers. Illustratively, the dormant lawn primer may be between 5 and 25 bp or between 7 and 20 bp or between 9 and 13 bp. In one example, the length of the dormant lawn primer is 9 bp, 10 bp or 13 bp. The use of shorter-length dormant primers, in addition to primers with a 3′ blocking group, not only prevents extension until following cluster generation but also prevents invasion (i.e. unwanted annealing), which would decrease amplification efficiency.

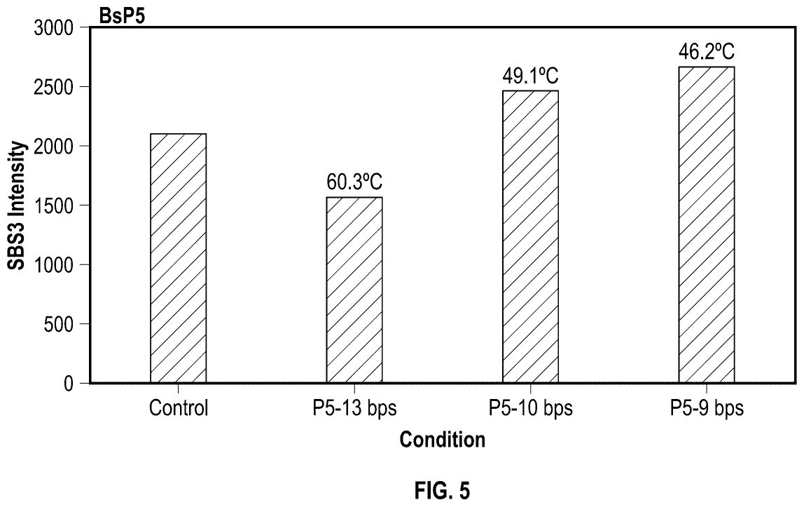

As shown in FIG. 5, if the blocked short primer is too long the Read 1 signal intensity drops off in parallel with an increase in the Tm of the primers. FIG. 5 shows that the decrease in R1 intensity when using BsP5 is probably due to unwanted annealing with the templates. However, further shortening of the length of BsP5 can be used to further lower the Tm and inhibit unwanted annealing. As depicted in FIG. 5, the BsP5 with 9 base pairs has a lower Tm and a higher signal intensity that the BsP5s with 10 base pairs and 13 base pairs.

In some examples, full-length P5 105 and P7 100 participate in the surface DNA clustering amplification, followed by CCL linearization to release the single strand for the sequencing of Read 1. After that, B-sP5 may be de-protected prior to PE turn re-synthesis. In some examples, the sP5 (B-sP5 after removal of blocking group B) may have a length of about 10 bps with relatively low melting temperature, so that amplification (e.g., bridge amplification or ExAmp), applied in the clustering step, may be used for the DNA strand hybridization and extension to fulfill the re-synthesis.

In some examples, the dormant lawn primer is grafted at a concentration in the range of about 0.2 μM to about 5 or about 0.4 μM to about 3 or about 0.5 μM to about 2.5 μM.

In a further example, the dormant lawn primer is grafted at a concentration of about 0.5 or about 1.1 or about 2.2 μM. In a preferred example, the dormant lawn primer is grafted at a concentration of about 2.2 μM. In some examples, the dormant lawn primer is blocked P5 primer, e.g., B-sP5, which includes a blocking group at its 3′ end and may be shorter than full-length P5.

In a further example, the dormant lawn primers may include or consist of a nucleic acid sequence selected from SEQ ID NO: 3, 4 or 5 or a variant thereof. The primers may also be blocked at the 3′ end (i.e. a 3′ blocking group), where the block prevents extension of the primer until the block is removed.

In a further aspect of the disclosure, there is provided a re-synthesis primer, the primer comprising a nucleic acid sequence selected from SEQ ID NO: 3, 4 or 5 or a variant thereof, and wherein the primer includes a 3′ blocking group that prevents extension of the primer until the blocking group is removed.

In one example the blocking group is a phosphate group. In one example the surface of the solid support is treated with a phosphatase to remove the block. Another example of the blocking group is a hairpin structure or other structure which provides steric hindrance at the 3′ end of the re-synthesis primer.

In another aspect of the disclosure there is provided a solid support for use in sequencing, wherein the support includes a plurality of lawn primers immobilised thereon and a plurality of dormant lawn primers immobilised thereon, wherein the dormant lawn primers include a blocking 3′ group that prevents extension until removed.

In one example, the lawn primers include a P7 primer and a P5 primer.

In another example, the dormant lawn primer a P5 primer. In a further example, the dormant lawn primer includes or consists of a nucleic acid sequence as defined in SEQ ID NO: 3, 4 or 5 or a variant thereof.

In one example, the ratio of lawn:dormant lawn primer ratio is selected from 5:1, 4:1, 3:1, 2:1, 1:1 and 1:2, 1:3, 1:4 and 1:5. In a preferred example, the ratio of lawn:dormant lawn primer ratio is selected from 2:1, 1:1 and 1:2.

In some examples, the lawn primers include unblocked P5 primers, blocked P5 primers, and unblocked P7 primers.

In another aspect, a method is provided that includes doping a plurality of P5 lawn primers, resulting in blocked P5 lawn primers (BsP5). In some examples, the BsP5 primers do not participate in cluster generation because they are blocked. In some examples, doping the P5 lawn primers results in blocking the 3′ end of the P5 lawn primers.

In some examples, doping the P5 lawn primers includes administering a solution to the P5 lawn primers that includes a single-stranded binding protein.

In some examples, BsP5 lawn primers are used in a nanowell of a flowcell as part of a sequencing reaction. In some examples, the single-stranded binding protein increases the tolerance for high levels of BsP5 lawn primers in a nanowell, without compromising sequencing metrics as defined percentage pass filter. In some examples, use of the single-stranded binding protein allows for at least 2,000 BsP5 lawn primers per nanowell, at least 5,000 BsP5 lawn primers per nanowell, at least 10,000 BsP5 primers per nanowell, or at least 15,000 BsP5 lawn primers per nanowell, without compromising sequencing metrics as defined by percentage pass filter.

In some examples, the single-stranded binding protein includes gp32. In some examples, the single-stranded binding protein includes any single-stranded binding protein described herein.

In some examples, the concentration of the single-stranded binding protein in the solution is between about 39 uM and about 45 uM. In some examples, a concentration of the single-stranded binding protein between about 39 uM and about 45 uM functions to prevent or inhibit consumption of the single-stranded binding protein by the BsP5 primers. In some examples, the concentration of the single-stranded binding protein in the solution formulation is between about 41 uM and about 43 uM. In some examples, a concentration of the single-stranded binding protein between about 41 uM and about 43 uM functions to prevent or inhibit consumption of the single-stranded binding protein by the BsP5 primers. In some examples, the concentration of the single-stranded binding protein in the solution formulation is about 42 uM. In some examples, a concentration of the single-stranded binding protein of about 42 uM functions to prevent or inhibit consumption of the single-stranded binding protein by the BsP5 primers.

In some examples, the solution further includes a recombinase. In some examples, the concentration of the recombinase in the solution is between about 5.6 uM and about 7.6 uM. In some examples, a concentration of recombinase in the solution between about 5.6 uM and about 7.6 uM functions to prevent or inhibit consumption of the recombinase by the BsP5 primers. In some examples, the concentration of the recombinase in the solution is about 6.6 uM. In some examples, a concentration of recombinase in the solution between of about 6.6 uM functions to prevent or inhibit consumption of the recombinase by the BsP5 primers.

In some examples, the solution further includes dNTPs that are used to extend a primer hybridised to a nucleic acid strand. In some examples, the solution further includes a polymerase, which is an enzyme used to synthesize a new strand of DNA by adding nucleotides to the new strand through complementary base-pairing to a template strand. In some examples, the solution includes the elements listed in Table 1. In some examples, the solution includes the elements listed in Table 2.

In another aspect, a method is provided of sequencing a target nucleic acid that includes doping a plurality of P5 lawn primers with a solution that includes a single-stranded binding protein. In some examples, the solution further includes a recombinase.

In some examples, the method of sequencing a target nucleic acid further includes immobilising doped BsP5 primers on a solid support. In some examples, the method of sequencing a target nucleic acid further includes immobilising P7 primers on the solid support. In some examples, the solid support is part of a nanowell within a flowcell.

In some examples, the method of sequencing a target nucleic acid further includes immobilising nucleic acid strands on the solid support. In some examples, the method of sequencing a target nucleic acid includes carrying out at least one sequencing read on the immobilised nucleic acid strands. In some examples, the method of sequencing a target nucleic acid includes carrying out two sequencing reads on the immobilised nucleic acid strands. In some examples, the BsP5 primers do not participate in the first sequencing read.

In another aspect, a method of doping primers is provided including doping a plurality of P5 lawn primers with a solution that includes a single-stranded binding protein. In some examples, the solution further includes a recombinase. In some examples, the concentration of the recombinase is between about 5.6 uM and about 7.6 uM and the concentration of the single-stranded binding protein is between about 41 uM and about 43 uM.

In some examples, the concentration of the recombinase is between about 5.6 uM and about 7.6 uM and the concentration of the single-stranded binding protein is less than about 41 uM. In some examples, the concentration of the single stranded binding protein is between about 41 uM and about 43 uM, and the concentration of the recombinase is less than about 5.6 uM,

### Bridge Amplification

Bridge amplification can occur on a flow cell. Single stranded template DNA is hybridised to lawn primers in a flow cell, and a polymerase is used to extend the primer to form double-stranded DNA. The double-stranded DNA is denatured, and the original template strand of the DNA molecule is washed away. This results in a single-stranded DNA molecule being bound to the lawn primers of the flow cell. The single-stranded DNA molecule turns over and forms a “bridge” by hybridising to a nearby lawn primer that is complementary to a sequence of the single-stranded DNA molecule. Polymerase extends the hybridised primer resulting in bridge amplification of the DNA molecule and the creation of a double-stranded DNA molecule. The double-stranded DNA molecule is then denatured resulting in two copies of single-stranded templates, one of which is immobilised to the support and the other of which may be washed away. The one which is immobilised as the support may be used in further bridge amplification operations so as to generate a cluster that subsequently may be sequenced.

### Exclusion Amplification

Exclusion amplification methods may allow for the amplification of a single target polynucleotide per substrate region and the production of a substantially monoclonal population of amplicons in a substrate region. For example, the rate of amplification of the first captured target polynucleotide within a substrate region may be more rapid relative to much slower rates of transport and capture of target polynucleotides at the substrate region. As such, the first target polynucleotide captured in a substrate region may be amplified rapidly and fill the entire substrate region, thus inhibiting the capture of additional target polynucleotide in the same substrate region. Alternatively, if a second target polynucleotide attaches to same substrate region after the first polynucleotide, the relatively rapid amplification of the first polynucleotide may fill enough of the substrate region to result in a signal that is sufficiently strong to perform sequencing by synthesis (e.g., the substrate region may be at least functionally monoclonal). The use of exclusion amplification may also result in super-Poisson distributions of monoclonal substrate regions; that is, the fraction of substrate regions in an array that are functionally monoclonal may exceed the fraction predicted by the Poisson distribution.

Increasing super-Poisson distributions of useful clusters is useful because more functionally monoclonal substrate regions may result in higher quality signal, and thus improved SBS; however, the seeding of target polynucleotides into substrate regions may follow a spatial Poisson distribution, where the trade-off for increasing the number of occupied substrate regions is increasing the number of polyclonal substrate regions. One method of obtaining higher super-Poisson distributions is to have seeding occur quickly, followed by a delay among the seeded target polynucleotide. The delay, termed “kinetic delay” because it is thought to arise through the biochemical reaction kinetics, gives one seeded target polynucleotide an earlier start over the other seeded targets. Exclusion amplification works by using recombinase to facilitate the invasion of primers (e.g., primers attached to a substrate region) into double-stranded DNA (e.g., a target polynucleotide) when the recombinase mediates a sequence match. The present compositions and methods may be adapted for use with recombinase to facilitate the invasion of the present capture primers and orthogonal capture primers into the present target polynucleotides when the recombinase mediates a sequence match. Indeed, the present compositions and methods may be adapted for use with any surface-based polynucleotide amplification methods such as thermal PCR, chemically denatured PCR, and enzymatically mediated methods (which may also be referred to as recombinase polymerase amplification (RPA), strand invasion, or ExAmp).

In some examples, exclusion amplification utilizes a solution that contains polymerase and dNTPs. In some examples, the solution further includes components capable of doping P5 lawn primers, resulting in BsP5 primers. In some examples, the solution includes a recombinase. In some examples, the solution includes a single-stranded binding protein.

In some examples, the concentration of recombinase in the solution is between about 1.0 uM and about 8.0 uM. In some examples, the concentration of recombinase in the solution is between about 1.5 uM and 7.0 uM. In some examples, the concentration of recombinase in the solution is about 1.5 uM, about 1.6 uM, about 1.7 uM, about 1.8 uM, about 1.9 uM, about 2.0 uM, about 2.1 uM, about 2.2 uM. about 2.3 uM, about 2.4 uM, about 2.5 uM, about 2.6 uM, about 2.7 uM, about 2.8 uM, about 2.9 uM, about 3.0 uM, about 3.1 uM, about 3.2 uM, about 3.3 uM, about 3.4 uM, about 3.5 uM, about 3.6 uM, about 3.7 uM, about 3.8 uM, about 3.9 uM, about 4.0 uM, about 4.1 uM, about 4.2 uM, about 4.3 uM, about 4.4 uM, about 4.5 uM, about 4.6 uM, about 4.7 uM, about 4.8 uM, about 4.9 uM, about 5.0 uM, about 5.1 uM, about 5.2 uM, about 5.3 uM, about 5.4 uM, about 5.5 uM, about 5.6 uM, about 5.7 uM, about 5.8 uM, about 5.9 uM, about 6.0 uM, about 6.1 uM, about 6.2 uM, about 6.3 uM, about 6.4 uM, about 6.5 uM, about 6.6 uM, about 6.7 uM, about 6.8 uM, about 6.9 uM, or about 7.0 uM.

In some examples, the concentration of recombinase in the solution is at or above about 5.6 uM and at or below about 7.6 uM. In some examples, a recombinase concentration at a range of between about 5.6 uM and 7.6 uM in the solution helps to maintain high sequencing metrics when the concentration of BsP5 primers is increased in the nanowells of the flow cells. In some examples, the high sequencing metrics is equivalent to a percentage pass filter of at least 70 in a sequencing run.

In some examples, a recombinase concentration in the solution at a range of between about 5.6 uM and 7.6 uM maintains high sequencing metrics when the number of BsP5 primers in a nanowell is at least 2,000, at least 5,000, at least 10,000, or at least 15,000. In some examples, a recombinase concentration in solution at a range of between about 5.6 uM and 7.6 uM maintains high sequencing metrics when the number of primers of BsP5 in a nanowell is greater than 15,000.

In some examples, the solution that is capable of doping P5 primers includes a single-stranded binding protein. In some examples, the concentration of the single-stranded binding protein in the solution is between about 10.0 uM and about 50.0 uM. In some examples, the concentration of the single-stranded binding protein in the solution is between about 15.0 uM and about 45.0 uM. In some examples, the concentration of the single-stranded binding protein in the solution is between about 35.0 uM and about 45.0 uM. In some examples, the concentration of the single-stranded binding protein in the solution is about 35.0 uM, about 35.1 uM, about 35.2 uM, about 35.3 uM, about 35.4 uM, about 35.5 uM, about 35.6 uM, about 35.7 uM, about 35.8 uM, about 35.9 uM, about 40.0 uM, about 40.1 uM, about 40.2 uM, about 40.3 uM, about 40.4 uM, about 40.5 uM, about 40.6 uM, about 40.7 uM, about 40.8 uM, about 40.9 uM, about 41.0 uM, about 41.1 uM, about 41.2 uM, about 41.3 uM, about 41.4 uM, about 41.5 uM, about 41.6 uM, about 41.7 uM, about 41.8 uM, about 41.9 uM, about 42.0 uM, about 42.1 uM, about 42.2 uM, about 42.3 uM, about 42.4 uM, about 42.5 uM, about 42.6 uM, about 42.7 uM, about 42.8 uM, about 42.9 uM, about 43.0 uM, about 43.1 uM, about 43.2 uM, about 43.3 uM, about 43.4 uM, about 43.5 uM, about 43.6 uM, about 43.7 uM, about 43.8 uM, about 43.9 uM, about 44.0 uM, about 44.1 uM, about 44.2 uM, about 44.3 uM, about 44.4 uM, about 44.5 uM, about 44.6 uM, about 44.7 uM, about 44.8 uM, about 44.9 uM, or about 45.0 uM.

In some examples, the concentration of a single-stranded binding protein in the solution is at or above about 39.0 uM and at or below about 45.0 uM. In some examples, a single-stranded binding protein at a range of between about 39.0 uM and 45.0 uM in the solution helps maintain high sequencing metrics when the concentration of BsP5 primers is increased in the nanowells of the flow cells. In some examples, the high sequencing metrics is equivalent to a percentage pass filter of at least 70 in a sequencing run.

In some examples, a single-stranded binding protein concentration in the solution at a range of between about 39.0 uM and about 45.0 uM maintains high sequencing metrics when the number of primers of BsP5 in a nanowell is at least 2,000, at least 5,000, at least 10,000, or at least 15,000. In some examples, a single-stranded binding protein concentration in the solution at a range of between about 39.0 uM and 45.0 uM maintains high sequencing metrics when the number of primers of BsP5 in a nanowell is greater than 15,000.

In some examples, the single-stranded binding protein used in the formulation capable of doping BsP5 primers includes gp32.

In some examples, the formulation that is capable of doping BsP5 primers is found in Tables 1 and 2.

### Nucleic Acids and Template Libraries

As will be understood by the skilled person, a double-stranded nucleic acid will typically be formed from two complementary polynucleotide strands made up of deoxyribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, also peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

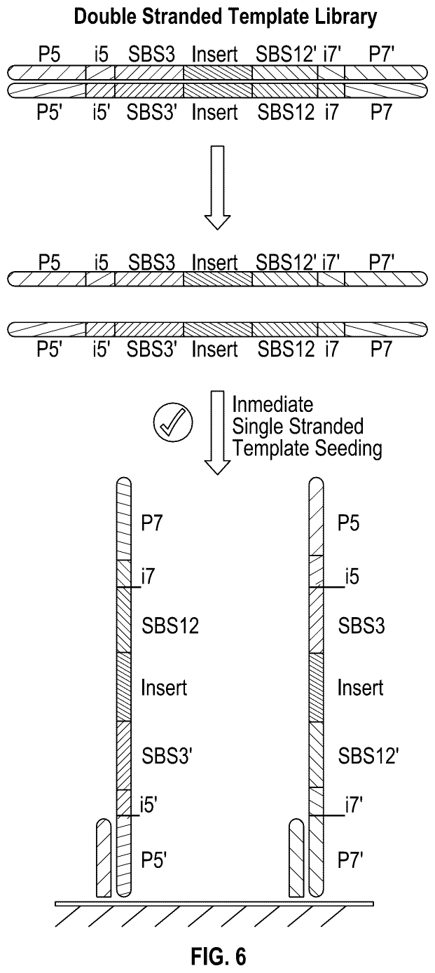

An example of a typical double-stranded nucleic acid template (which may be provided in a library of such templates) is shown in FIG. 6. In one example, a first strand of the template includes, in the 5′ to 3′ direction, a first lawn primer-binding sequence (e.g., P5), an index sequence (e.g., i5), a first sequencing primer binding site (e.g., SBS3), an insert corresponding to the template DNA to be sequenced, a second sequencing primer binding site (e.g. SBS12′), a second index sequence (e.g. i7′) and a second lawn primer-binding sequence (e.g. the complement of P7). The second strand of the template includes, in the 3′ to 5′ direction, a first lawn primer-binding site (e.g. the complement of P5), an index sequence (e.g. i5′, which is complementary to i5), a first sequencing primer binding site (e.g. SBS3′ which is complementary to SBS3), an insert corresponding to the complement of the template DNA to be sequenced, a second sequencing primer binding site (e.g. SBS12, which is complementary to SBS12), a second index sequence (e.g. i7, which is complementary to i7) and a second lawn primer-binding sequence (e.g. P7). Either template is referred to herein as a “template strand” or “a single stranded template”. Both template strands annealed together as shown in FIG. 1, is referred to herein as “a double stranded template”. The combination of a primer-binding sequence, an index sequence and a sequencing binding site is referred to herein as an adaptor sequence, and a single insert is flanked by a 5′ adaptor sequence and a 3′ adaptor sequence. The first primer-binding sequence may also include a sequencing primer for the index read (i5).

In one example, the primer-binding sequences of the adaptors are complementary to short primer sequences (or lawn primers) present on the surface of the flow cells. Binding of suitable portions of the adaptors to their complements (P5 and P7) on—for example—the surface of the flow cell, permits nucleic acid amplification. As used herein “′” denotes the complementary strand.

The primer-binding sequences in the adaptor which permit hybridisation to amplification (lawn) primers will typically be around 20-40 nucleotides in length, although, in examples, the disclosure is not limited to sequences of this length. The precise identity of the amplification primers, and hence the cognate sequences in the adaptors, are generally not material to the disclosure, as long as the primer-binding sequences are able to interact with the amplification primers in order to direct amplification. The sequence of the amplification primers may be specific for a particular target nucleic acid that it is desired to amplify, but in other examples these sequences may be “universal” primer sequences which enable amplification of any target nucleic acid of known or unknown sequence which has been modified to enable amplification with the universal primers. The criteria for design of PCR primers are generally well known to those of ordinary skill in the art. “Primer-binding sequences” may also be referred to as “clustering sequences” “clustering primers” or “cluster primers” in the present disclosure, and such terms may be used interchangeably.

The index sequences (also known as a barcode or tag sequence) are unique short DNA sequences that are added to each DNA fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analyzed in a single run, without drastically increasing run cost or run time. Examples of tag sequences are found in WO05068656, the entire contents of which are incorporated by reference herein. The tag can be read at the end of the first read, or equally at the end of the second read. The disclosure is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example US60/899221, the entire contents of which are incorporated by reference herein. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing binding sites are sequencing and/or index primer binding sites and indicates the starting point of the sequencing read. During the sequencing process, a sequencing primer anneals (i.e. hybridises) to a portion of the sequencing binding site on the template strand. The DNA polymerase enzyme binds to this site and incorporates complementary nucleotides base by base into the growing opposite strand. In one example, the sequencing process includes a first and second sequencing read. The first sequencing read may include the binding of a first sequencing primer (read 1 sequencing primer) to the first sequencing binding site (e.g., SBS3′) followed by synthesis and sequencing of the complementary strand. This leads to the sequencing of the insert. In a second step, an index sequencing primer (e.g. i7 sequencing primer) binds to a second sequencing binding site (e.g. SBS12) leading to synthesis and sequencing of the index sequence (e.g. sequencing of the i7 primer). The second sequencing read may include binding of an index sequencing primer (e.g. i5 sequencing primer) to the complement of the first sequencing binding site on the template (e.g. SBS3) and synthesis and sequencing of the index sequence (e.g. i5). In a second step, a second sequencing primer (read 2 sequencing primer) binds to the complement of the primer (e.g. i7 sequencing primer) binds to a second sequencing binding site (e.g. SBS12′) leading to synthesis and sequencing of the insert in the reverse direction.

Once a double stranded nucleic acid template library is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al). In one example, chemical denaturation, such as NaOH or formamide, is used. Suitable denaturation agents include: acidic nucleic acid denaturants such as acetic acid, HCl, or nitric acid; basic nucleic acid denaturants such as NaOH; or other nucleic acid denaturants such as DMSO, formamide, betaine, guanidine, sodium salicylate, propylene glycol or urea. Preferred denaturation agents are formamide and NaOH, preferably formamide.

As illustrated in FIG. 6, following denaturation, a single-stranded template library is in one example contacted in free solution onto a solid support comprising surface capture moieties (for example full-length P5 and P7 lawn primers). This solid support is typically a flowcell, although in alternative examples, seeding and clustering can be conducted off-flowcell using, for example, microbeads or the like. The solid support further may include dormant lawn primers, e.g., BsP5 lawn primers, which may be used for PE resynthesis in a manner such as described elsewhere herein.

### Solid Supports

The disclosure may make use of solid supports made up of a substrate or matrix (e.g. glass slides, polymer beads etc) which has been “functionalised”, for example by application of a layer or coating of an intermediate material comprising reactive groups which permit covalent attachment to biomolecules, such as polynucleotides. Examples of such supports include, but are not limited to, a substrate such as glass. In such examples, the biomolecules (e.g. polynucleotides) may be directly covalently attached to the intermediate material but the intermediate material may itself be non-covalently attached to the substrate or matrix (e.g. the glass substrate). The term “covalent attachment to a solid support” is to be interpreted accordingly as encompassing this type of arrangement. Alternatively, the substrate such as glass may be treated to permit direct covalent attachment of a biomolecule; for example, glass may be treated with hydrochloric acid, thus exposing the hydroxyl groups of the glass, and phosphite-triester chemistry used to directly attach a nucleotide to the glass via a covalent bond between the hydroxyl group of the glass and the phosphate group of the nucleotide.

In other examples, the solid support may be “functionalised” by application of a layer or coating of an intermediate material comprising groups that permit non-covalent attachment to biomolecules. In such examples, the groups on the solid support may form one or more of ionic bonds, hydrogen bonds, hydrophobic interactions, 7C-7C interactions, van der Waals interactions and host-guest interactions, to a corresponding group on the biomolecules (e.g. polynucleotides). The interactions formed between the group on the solid support and the corresponding group on the biomolecules may be configured to cause immobilisation or attachment under the conditions in which it is intended to use the support, for example in applications requiring nucleic acid amplification and/or sequencing. For example, the interactions formed between the group on the solid support and the corresponding group on the biomolecules may be configured such that the biomolecules remain attached to the solid support during amplification and/or sequencing.

In other examples, the solid support may be “functionalised” by application of an intermediate material comprising groups that permit attachment via metal-coordination bonds to biomolecules. In such examples, the groups on the solid support may include ligands (e.g. metal-coordination groups), which are able to bind with a metal moiety on the biomolecule. Alternatively, or in addition, the groups on the solid support may include metal moieties, which are able to bind with a ligand on the biomolecule. The metal-coordination interactions formed between the ligand and the metal moiety may be configured to cause immobilisation or attachment of the biomolecule under the conditions in which it is intended to use the support, for example in applications requiring nucleic acid amplification and/or sequencing. For example, the interactions formed between the group on the solid support and the corresponding group on the biomolecules may be configured such that the biomolecules remain attached to the solid support during amplification and/or sequencing.

When referring to immobilisation or attachment of molecules (e.g. nucleic acids) to a solid support, the terms “immobilised” and “attached” are used interchangeably herein and both terms are intended to encompass direct or indirect, covalent or non-covalent attachment, unless indicated otherwise, either explicitly or by context. In certain examples of the disclosure, covalent attachment may be preferred; in other examples, attachment using non-covalent interactions may be preferred; in yet other examples, attachment using metal-coordination bonds may be preferred. However, in general the molecules (e.g. nucleic acids) remain immobilised or attached to the support under the conditions in which it is intended to use the support, for example in applications requiring nucleic acid amplification and/or sequencing. When referring to attachment of nucleic acids to other nucleic acids, then the terms “immobilised” and “hybridised” are used herein, and generally refer to hydrogen bonding between complementary nucleic acids.

If the amplification is performed on beads, either with a single or multiple extendable primers, the beads may be analysed in solution, in individual wells of a microtitre or picotitre plate, immobilised in individual wells, for example in a fibre optic type device, or immobilised as an array on a solid support. The solid support may be a planar surface, for example a microscope slide, wherein the beads are deposited randomly and held in place with a film of polymer, for example agarose or acrylamide.

### Amplification and Sequencing of Template Strands

Once a library comprising template nucleotide strands has been prepared, the templates are seeded onto a solid support and then amplified to generate a cluster of single template molecules.

By way of brief example, following attachment of the P5 and P7 primers (in addition to dormant lawn primers, e.g., BsP5), the solid support may be contacted with the template to be amplified under conditions which permit hybridisation (or annealing—such terms may be used interchangeably) between the template and the immobilised primers (also referred to herein as “lawn primers”). The template is usually added in free solution under suitable hybridisation conditions, which will be apparent to the skilled reader. Typically, hybridisation conditions are, for example, 5×SSC at 40° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e. the complement of either P5 or P7) which in some methods is capable of bridging to the second primer molecule immobilised on the solid support and binding. In this method, further rounds of amplification (analogous to a standard PCR reaction) lead to the formation of clusters or colonies of template molecules bound to the solid support. Thus, in this example, solid-phase amplification by either the method analogous to that of WO 98/44151 or that of WO 00/18957 (the contents of which are incorporated herein in their entirety by reference) will result in production of a clustered array that includes colonies of “bridged” amplification products. Both strands of the amplification products will be immobilised on the solid support at or near the 5′ end, this attachment being derived from the original attachment of the amplification primers. Typically, the amplification products within each colony will be derived from amplification of a single template (target) molecule. Other amplification procedures may be used, and will be known to the skilled person. For example, amplification may be isothermal amplification using a strand displacement polymerase; or may be exclusion amplification as described in WO 2013/188582, the entire contents of which are incorporated by reference herein. The method may also involve a number of rounds of invasion by a competing immobilised primer (or lawn primer) and strand displacement of the template to the competing primer. Further information on amplification can be found in WO0206456 and WO07107710, the entire contents of each of which are incorporated by reference herein. Through such approaches, a cluster of single template molecules is formed.

To facilitate sequencing, it is preferable if one of the strands is removed from the surface to allow efficient hybridisation of a sequencing primer to the remaining immobilised strand. Suitable methods for linearisation are described in more detail in application number WO07010251, the entire contents of which are incorporated by reference herein. The T* sites (allyl T, also referred to as CCL) indicated in SEQ ID NOS: 6-11 may be used to linearize full-length P5 after the initial amplification step. Because such sites are within the poly-T spacer, it is expected that the remaining bases of P5 following such linearization substantially will not interfere with subsequent PE resynthesis using deblocked BsP5 in a manner such as described elsewhere herein.

Sequence data can be obtained from both ends of a template duplex by obtaining a sequence read from one strand of the template from a primer in solution, copying the strand using immobilised primers, releasing the first strand and sequencing the second, copied strand.

For example, sequence data can be obtained from both ends of the immobilised duplex by a method wherein the duplex is treated to free a 3′-hydroxyl moiety that can be used an extension primer. The extension primer can then be used to read the first sequence from one strand of the template. After the first read, the strand can be extended to fully copy all the bases up to the end of the first strand. This second copy remains attached to the surface at the 5′ -end. If the first strand is removed from the surface, the sequence of the second strand can be read. This gives a sequence read from both ends of the original fragment. The process whereby the strand is regenerated after the first read is known as “Paired-end resynthesis” or “PE resynthesis”. The typical steps of pairwise sequencing are known and have been described in WO 2008/041002, the entire contents of which are incorporated by reference herein.

Sequencing can be carried out using any suitable “sequencing-by-synthesis” technique, wherein nucleotides are added successively to the free 3′ hydroxyl group, resulting in synthesis of a polynucleotide chain in the 5′ to 3′ direction. The nature of the nucleotide added is preferably determined after each addition. One particular sequencing method relies on the use of modified nucleotides that can act as reversible chain terminators. Such reversible chain terminators include removable 3′ blocking groups. Once such a modified nucleotide has been incorporated into the growing polynucleotide chain complementary to the region of the template being sequenced there is no free 3′-OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application PCT/GB/2007/001770, the entire contents of which are incorporated by reference herein. Alternatively, a separate reaction may be carried out including each of the modified nucleotides added individually.

The modified nucleotides may carry a label to facilitate their detection. In a particular example, the label is a fluorescent label. Each nucleotide type may carry a different fluorescent label. However the detectable label need not be a fluorescent label. Any label can be used which allows the detection of the incorporation of the nucleotide into the DNA sequence. One method for detecting the fluorescently labelled nucleotides includes using laser light of a wavelength specific for the labelled nucleotides, or the use of other suitable sources of illumination. The fluorescence from the label on an incorporated nucleotide may be detected by a CCD camera or other suitable detection means. Suitable detection means are described in PCT/US2007/007991, the entire contents of which are incorporated by reference herein.

Alternative methods of sequencing include sequencing by ligation, for example as described in U.S. Pat. No. 6,306,597 or WO06084132, the entire contents of each of which are incorporated by reference herein.

An extension reaction, in which nucleotides are added to the 3′ end of a primer is performed using a polymerase, such as a DNA or RNA polymerase. In one example, the polymerase is a non-thermal isothermal strand displacement polymerase. Suitable non-thermostable strand displacement polymerases according to the present disclosure can be found, for example, through New England BioLabs, Inc. and include phi29, Bsu, Klenow, DNA Polymerase I (E. coli), and Therminator. A particularly preferred polymerase is Bsu.

Reference to P5 and P7 could refer to different primer sequences. Any suitable primer sequence combinations are encompassed by the present disclosure.

### WORKING EXAMPLE

The following example is intended to be purely illustrative, and not limiting in any way.

### Example 1: Improved Sequencing Data Quality Through Use of a Formulation Containing Higher Concentrations of Single-Stranded Binding Protein and Recombinase

In this experiment, sequencing metrics were compared for co-grafted clustering primers P7 and paired-end blocked short P5 primers, between a standard clustering workflow and non-bridging clustering (NBC). Two different doping formulations were used (TCX formulation and Ras6T formulation), which are shown in Table 1 (TCX formulation) and Table 2 (Ras6T formulation).

TABLE 1
(TCX formulation)

| Component | V1 | Unit |
|---|---|---|
| 1M Tris, pH 8.6 | 0.025 | M |
| ATP, Solid | 5 | mM |
| 0.5M TCEP | 4 | mM |
| Creatine Phosphate | 0.09 | M |
| dNTP Pool | 2.4 | mM |
| Mannitol | 2 | % |
| HPBCD | 7.5 | % |
| Trehalose dihydrate | 7.5 | % |
| Creatine Kinase (CK) | 0.2 | mg/ml |
| GP32.v1.5 | 1.6 | mg/ml |
| | 42 | uM |
| Bacillus subtilis (Bsu) DNA | 1.044 | mg/ml |
| Pol.V3 | | |
| Rec233.V2 (recombinase) | 0.25 | mg/ml |
| | 6.6 | uM |
| UvsY.V1 | 0.032 | mg/ml |

TABLE 2
(Ras6T formulation)

| Component | V1 | Unit |
|---|---|---|
| 1M Tris, pH 8.6 | 25 | mM |
| TCEP | 2 | mM |
| KPC | 50 | mM |
| dNTP | 1.2 | mM |
| ATP | 2.5 | mM |
| Creatine Kinase (CK) | 1 | X |
| BSU | 11.54 | U/uL |
| gp32 | 18 | uM |
| UvsY | 1 | uM |
| Rec233 (recombinase) | 2 | uM |
| MgOAc | 8 | mM |

One of the major differences between these formulations is that relative to the Ras6T formulation, the TCX formulation has higher concentrations of the single-strand binding protein gp32 and the recombinase.

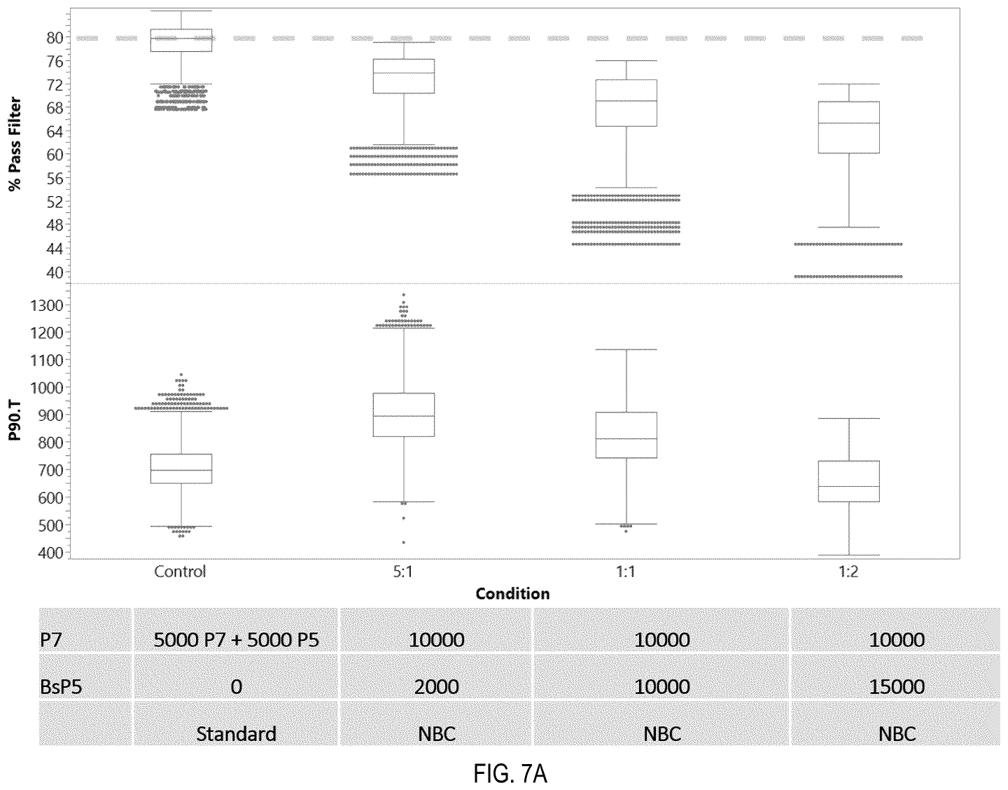

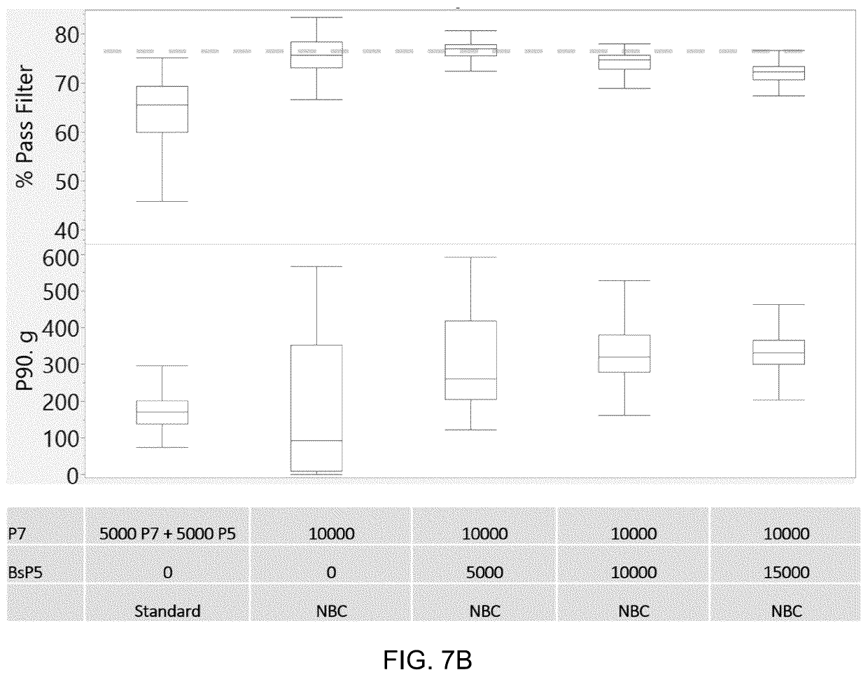

When the Ras6T formulation was tested, it was applied to the sample using one push for 60 minutes, under isothermal conditions. When the TCX formulation was tested, it was applied to the sample using three pushes, under isothermal conditions. Each push occurred for 30 minutes. Results from these experiments are shown in FIGS. 7A (application of the Ras6T formulation) and 7B (application of the TCX formulation).

Comparing the data in FIGS. 7A and 7B indicates that the TCX formulation has a higher tolerance of BsP5 primers per nanowell. In particular, as shown in FIG. 7B (application of the TCX formulation), the percent pass filter for the NBC was higher than the standard clustering workflow even when 15,000 primers per nanowell. This finding can be compared to FIG. 7A (application of the Ras6T formulation) in which there is a precipitous decline in the percent pass filter in the NBC relative to the standard clustering workflow, when the BsP5 primers per nanowell reached 10,000 and 15,000. Note that the percent pass filter is a measure of the quality of the sequencing run. A percent pass filter of 70% or higher indicates a successful sequencing run.

This finding that the TCX formulation can tolerate a higher level BsP5 primers per nanowell may be due to the higher concentration of the single-stranded binding protein gp32 and the recombinase.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

SEQUENCE LISTING

SEQ ID NO: 1 (normal P5)
/5Hexynyl/TTTTTTAATGATACGGCGACCACCGAGA/ideoxyU/
CTACAC
SEQ ID No: 2 (full length P7)
/5Hexynyl/TTTTTTCAAGCAGAAGACGGCATAC/18oxodG/AGAT
SEQ ID NO: 3 BsP5 (13)
TTTTTTGGCGACCACCGAG
SEQ ID NO: 4 BsP5(10)
TTTTTTTACGGCGACC
SEQ ID NO: 5 BsP5 (9)
TTTTTTTACGGCGAC
SEQ ID NO: 6 Full-length P5, option 1
TTTTTT*AATGATACGGCGACCACCGAGATCTACAC
SEQ ID NO: 7 Full-length P5, option 2
TTTTT*TAATGATACGGCGACCACCGAGATCTACAC
SEQ ID NO: 8 Full-length P5, option 3
TTTT*TTAATGATACGGCGACCACCGAGATCTACAC
SEQ ID NO: 9 Full-length P5, option 4
TTT*TTTAATGATACGGCGACCACCGAGATCTACAC
SEQ ID NO: 10 Full-length P5, option 5
TT*TTTTAATGATACGGCGACCACCGAGATCTACAC
SEQ ID NO: 11 Full-length P5, option 6
T*TTTTTAATGATACGGCGACCACCGAGATCTACAC
T*: Linearizable T (ally1 T)
T: previous position of U for linearization in normal P5 (SEQ ID NO: 1)

1. A method of sequencing a target nucleic acid sequence, wherein the method comprises:
a. providing a solid support having immobilised thereon a cluster of first immobilised nucleic acid strands including a target nucleic acid sequence, wherein the solid support has a plurality of dormant lawn primers, wherein the dormant lawn primers are blocked at the 3′ end;
b. carrying out a first sequencing read to determine the sequence of a region of the first immobilised strands, preferably by a sequencing-by-synthesis technique or by a sequencing-by ligation technique;
c. removing the blocking groups from the dormant primers to allow extension from the 3′ ends of the unblocked primers;
d. carrying out a second extension reaction to extend the unblocked primer using the immobilised strand as a template to generate a cluster of second immobilised nucleic acid strands;
e. carrying out a second sequencing read to determine the sequence of a region of the second immobilised strands, preferably by a sequencing-by-synthesis technique or by a sequencing-by ligation technique;
wherein determining the sequences of the regions of the first and second immobilised strands achieves pairwise sequencing of said target nucleic acid sequence.

2. The method of claim 1, wherein the blocking group is a phosphate group that is bound to the 3′ end of the dormant lawn primers.

3. The method of claim 1, further comprising at least one step of amplifying the immobilised target nucleic acids.

4. A solid support for use in sequencing, wherein the support comprises a plurality of lawn primers immobilised on the solid support, and a plurality of dormant lawn primers immobilised on the solid support, wherein the dormant lawn primers comprise a blocking 3′ group that prevents extension until removed.

5. The solid support of claim 4, wherein the plurality of lawn primers comprises a P5 primer and a P7 primer.

6. The solid support of claim 5, wherein P5 comprises a nucleic acid sequence as defined in any one of SEQ ID NO: 6, SEQ ID NO: 7, SEQ ID NO: 8, SEQ ID NO: 9, SEQ ID NO: 10, or SEQ ID NO: 11, or a variant thereof, and wherein P7 comprises a nucleic acid sequence as defined in SEQ ID NO: 2 or a variant thereof.

7. The solid support of claim 5, wherein P5 is between 10 and 50 pb, and wherein P7 is between 10 bp and 50 bp.

8. The solid support of claim 5, wherein P5 is between 5 and 25 bp, and wherein P7 or the variant thereof is between 5 bp and 25 bp.

9. The solid support of claim 5, wherein P5 is 9 bp, 10 bp or 13 bp, and wherein P7 is 9 pb, 10 bp, or 13 bp.

10. The solid support of claim 4, wherein the plurality of dormant lawn primer comprises a BsP5 primer.

11. The solid support of claim 4, wherein the dormant lawn primers comprise at least 80%, at least 85%, at least 90%, or at least 95% of any of SEQ ID NO: 3, SEQ ID NO: 4, or SEQ ID NO: 5.

12. The solid support of claim 4, wherein the dormant lawn primers are blocked at the 3′ end by a phosphate group.

13. The solid support of claim 4, wherein the lawn:dormant lawn primer ratio is selected from about 5:1, about 4:1, about 3:1, about 2:1, about 1:1, about 1:2, about 1:3, about 1:4, and about 1:5.

14. A solid support comprising a plurality of lawn primers comprising at least one P5 primer and at least one P7 primer, and a plurality of dormant lawn primers comprising at least one P5 primer.

15. The solid support of claim 14, wherein at least one dormant lawn P5 primer is blocked at the 3′ end.

16. The solid support of claim 15, wherein at least one of the P5 primers is blocked at the 3′ end with a phosphate group.

17. The solid support of claim 14, wherein the at least one P7 lawn primer, the least one P5 lawn primer, and the least one P5 dormant lawn primer exist in approximately the following ratio: 1 (P7 lawn primer): 1 (P5 lawn primer): 2 (P5 dormant lawn primer).

18. The solid support of claim 14, wherein the at least one P5 dormant lawn primer comprises at least 80%, at least 85%, at least 90%, or at least 95% sequence identity to any of SEQ ID NO 3; SEQ ID NO: 4, or SEQ ID NO: 5.

19.-39. (canceled)

40. A method of sequencing a target nucleic acid, comprising:
a. generating an immobilised cluster of first nucleic acid strands on a solid support, wherein a plurality of p7 lawn primers and a plurality of blocked P5 lawn primers are immobilised on the solid support, and
wherein the plurality of blocked P5 lawn primers have been doped with a solution comprising a single-stranded binding protein;
b. carrying out a first sequencing read to determine a sequence of a region of the first immobilised nucleic acid strands;
c. removing the blocking groups from the P5 primers to allow extension from the 3′ ends of the unblocked P5 primers;
d. carrying out a second extension reaction to extend the unblocked P5 primers using the immobilised nucleic strands as a template to generate a cluster of second immobilised nucleic acid strands;
e. carrying out a second sequencing read to determine a sequence of a region of the second immobilised strands;
wherein determining the sequences of the regions of the first and second immobilised nucleic acid strands achieves pairwise sequencing of said target nucleic acid sequence.

41. The method of claim 40, wherein the single-stranded binding protein comprises gp32.

42. The method of claim 40, wherein the concentration of the single-stranded binding protein in the solution is greater than or equal to 39 uM and less than or equal to 45 uM.

43. The method of claim 40, wherein the solution further comprises recombinase.

44. The method of claim 43, wherein the concentration of recombinase is greater than or equal to 5.6 uM and less than or equal to 7.6 uM.

45.-54. (canceled)


SEQUENCE LISTING (11 sequences)

SEQ ID NO: 1 - DNA, 35 bp, synthetic construct
  source 1..35: mol_type = other DNA
  modified_base 1: mod_base = OTHER; note = 5Hexynyl-T
  modified_base 29: mod_base = OTHER; note = ideoxyU
  misc_feature 1..35: note = normal P5
ttttttaatgatacggcgaccaccgaganctacac

SEQ ID NO: 2 - DNA, 30 bp, synthetic construct
  source 1..30: mol_type = other DNA
  modified_base 1: mod_base = OTHER; note = 5Hexynyl-T
  modified_base 26: mod_base = OTHER; note = i8oxodG
  misc_feature 1..30: note = full length P7
ttttttcaagcagaagacggcatacnagat

SEQ ID NO: 3 - DNA, 19 bp, synthetic construct
  source 1..19: mol_type = other DNA
  misc_feature 1..19: note = BsP5 (13)
ttttttggcgaccaccgag

SEQ ID NO: 4 - DNA, 16 bp, synthetic construct
  source 1..16: mol_type = other DNA
  misc_feature 1..16: note = BsP5(10)
tttttttacggcgacc

SEQ ID NO: 5 - DNA, 15 bp, synthetic construct
  source 1..15: mol_type = other DNA
  misc_feature 1..15: note = BsP5 (9)
tttttttacggcgac

SEQ ID NO: 6 - DNA, 35 bp, synthetic construct
  source 1..35: mol_type = other DNA
  misc_feature 1..35: note = Full-length P5, option 1
  misc_feature 6: note = Linearizable T (allyl T)
  misc_feature 29: note = previous position of U for linearization in normal P5 (SEQ ID NO: 1)
ttttttaatgatacggcgaccaccgagatctacac

SEQ ID NO: 7 - DNA, 35 bp, synthetic construct; identical to SEQ ID NO: 6
  source 1..35: mol_type = other DNA
  misc_feature 1..35: note = Full-length P5, option 2
  misc_feature 5: note = Linearizable T (allyl T)
  misc_feature 29: note = previous position of U for linearization in normal P5 (SEQ ID NO: 1)

SEQ ID NO: 8 - DNA, 35 bp, synthetic construct; identical to SEQ ID NO: 6
  source 1..35: mol_type = other DNA
  misc_feature 1..35: note = Full-length P5, option 3
  misc_feature 4: note = Linearizable T (allyl T)
  misc_feature 29: note = previous position of U for linearization in normal P5 (SEQ ID NO: 1)

SEQ ID NO: 9 - DNA, 35 bp, synthetic construct; identical to SEQ ID NO: 6
  source 1..35: mol_type = other DNA
  misc_feature 1..35: note = Full-length P5, option 4
  misc_feature 3: note = Linearizable T (allyl T)
  misc_feature 29: note = previous position of U for linearization in normal P5 (SEQ ID NO: 1)

SEQ ID NO: 10 - DNA, 35 bp, synthetic construct; identical to SEQ ID NO: 6
  source 1..35: mol_type = other DNA
  misc_feature 1..35: note = Full-length P5, option 5
  misc_feature 2: note = Linearizable T (allyl T)
  misc_feature 29: note = previous position of U for linearization in normal P5 (SEQ ID NO: 1)

SEQ ID NO: 11 - DNA, 35 bp, synthetic construct; identical to SEQ ID NO: 6
  source 1..35: mol_type = other DNA
  misc_feature 1..35: note = Full-length P5, option 6
  misc_feature 1: note = Linearizable T (allyl T)
  misc_feature 29: note = previous position of U for linearization in normal P5 (SEQ ID NO: 1)